(19) United States
(12) Patent Application Publication
(10) Pub. No.: US 2019/0269362 A1
(43) Pub. Date: Sep. 5, 2019

(54) DEVICE AND METHOD FOR USER-ACTUATED MIXING OF DRY REAGENT WITH LIQUID

(71) Applicant: Milo Sensors, Inc., Santa Barbara, CA (US)

(72) Inventors: Shari Howard, Santa Barbara, CA (US); Evan Strenk, Santa Barbara, CA (US); Bob Lansdorp, Vancouver (CA); Michael Williams, Santa Barbara, CA (US)

(21) Appl. No.: 15/968,994

(22) Filed: May 2, 2018

Related U.S. Application Data
(60) Provisional application No. 62/636,890, filed on Mar. 1, 2018.

(51) Int. Cl. A61B 5/00; A61B 5/1486

(52) U.S. Cl. CPC A61B 5/4266; A61B 5/1486; A61B 5/6804

(57) ABSTRACT

A device for a user-actuated mixing mechanism includes a cartridge with a dry reagent compartment, a liquid reservoir, and a fluidic barrier. The fluidic barrier is capable of separating the dry reagent compartment from the liquid reservoir. The dry reagent compartment may contain at least one lyophilized enzyme and the liquid reservoir may contain at least one liquid reagent. The liquid reservoir is capable of receiving a target analyte from secretions of a test subject using the device. When the fluidic barrier is removed from the cartridge, the dry reagent compartment is in contact with the liquid reservoir such that the lyophilized enzyme is capable of reacting with the target analyte from the test subject.

### CROSS-REFERENCE TO RELATED APPLICATIONS

The application claims priority to and benefit of U.S. Provisional Patent Application No. 62/636,890 and U.S. Provisional Patent Application No. 62/500,414, the content of which are incorporated by reference in its entirety.

### FIELD OF THE INVENTION

The present invention relates to devices that separately store and mix two components by a user-actuated mixing mechanism. More specifically, the invention relates to a transdermal analyte sensing device for long-term storage of dry reagents and liquids that enables the mixing of dry reagents, such as lyophilized enzymes, with liquids upon the user-actuated mechanism to facilitate the function of the sensing device.

### BACKGROUND OF THE INVENTION

Devices that store components of a mixture separately are well known in many fields. Such devices are capable of protecting reagents from exposure to air or atmospheric moisture before the components are mixed. However, these devices do not allow for the ease of mixing of two separate components for a specific purpose such as medical or scientific testing.

For example, it is known that some reagents are unstable in liquid, but are stable in dry storage. In the field of electrochemical sensors, glucose strips are commonly employed to measure glucose in blood. A drop of blood is often used to hydrate enzymes of glucose strips, such as glucose oxidase, that is stored in a lyophilized form until mixing with liquids, such as ethanol.

In the case of transdermal analyte sensing devices, hydration of enzymes by body fluid or biofluids, such as sweat, is troublesome. In this situation, the flow of the liquid cannot be relied upon in a transdermal analyte sensing device because the liquid is mixed with biofluids of a subject. Therefore, there exists a need for a device that stores dry reagents and liquid separately, mixes upon a user-actuated mixing mechanism, and thereby allows transdermal analyte sensing devices to have an extended shelf-life with hydrated reagents upon activation.

It is an object of the invention to provide a long-term storage solution for dry reagents of the transdermal analyte sensing device.

It is an object of the invention to provide a transdermal analyte sensing device having two compartments to store dry reagents and liquid separately.

It is an object of the invention to provide a transdermal analyte sensing device having a user-actuated mixing mechanism for the mixture of dry reagents and liquid before the sensing device is in use.

### SUMMARY OF THE INVENTION

The present invention is a device that has two compartments for the storage of two reagents separately until the time they are mixed by a user-actuated mixing mechanism.

In one particular embodiment, the invention is a transdermal analyte sensing device capable of storing dry reagents and liquid reagents in separate compartments. The user mixes the dry reagents and liquid reagents upon user actuation of a mixing mechanism. The transdermal analyte sensing device according to the present invention comprises a cartridge with a hinge feature, which includes a dry reagent compartment, a liquid reservoir, and at least one fluidic barrier. The fluidic barrier is capable of separating the dry reagent compartment from the liquid reservoir. In an embodiment, the dry reagent compartment has at least one lyophilized enzyme, such alcohol oxidase, and the liquid reservoir has at least one liquid reagent, such as hydrogel.

When the transdermal sensing device is placed on a test subject, such as the subject's wrist in contact with the skin, the liquid reservoir is capable of receiving a target analyte from secretions, such as sweat or insensible perspiration, of the test subject and diffusing the target analyte into the liquid in the liquid reservoir.

In one embodiment of the transdermal analyte sensing device, there is a user-actuated mixing method comprising of a fluidic barrier separating the two compartments. When the fluidic barrier is removed from the cartridge, the dry reagent compartment comes in contact with the liquid reservoir such that the lyophilized enzyme is capable of reacting with the target analyte in the liquid reservoir. The cartridge further has a snap feature that is capable of keeping the dry reagent compartment in direct contact with the liquid reservoir by keeping the two compartments tightly closed.

These and other objects, advantages, and features of the invention will be more fully understood and appreciated by reference to the detailed description of the current embodiments and the drawings.

### BRIEF DESCRIPTION OF THE DRAWINGS

FIG. 1 is a side cross-sectional view of a cartridge in accordance with an embodiment of the present invention.

FIG. 2 is an exploded view of the cartridge in accordance with an embodiment of the present invention.

FIG. 3A is a side perspective view of the cartridge in accordance with an embodiment of the present invention, having a fluidic barrier attached to the cartridge.

FIG. 3B illustrates a side perspective view of the cartridge, where the fluidic barrier was pulled out from the cartridge.

FIG. 3C is a side perspective view of the cartridge in accordance with an embodiment of the present invention.

FIG. 4A is a top perspective view of the cartridge in accordance with an embodiment of the present invention.

FIG. 4B is a top perspective view of the cartridge in accordance with an embodiment of the present invention.

FIG. 5A is a perspective view of a transdermal analyte sensing device in accordance with an embodiment of the present invention having the cartridge attached with a wristband.

FIG. 5B is a side cross-sectional view of the transdermal analyte sensing device in accordance with an embodiment of the present invention.

### DETAILED DESCRIPTION OF EMBODIMENTS

The invention will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and to fully convey the scope of the invention to those skilled in the art.

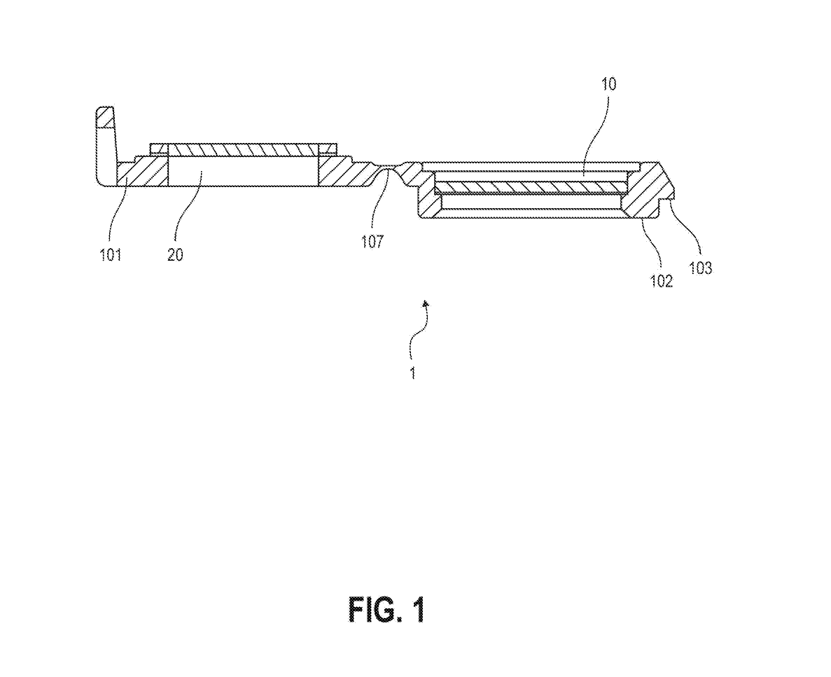

As shown in FIG. 1, a cartridge 1 according to an embodiment of the invention is comprised of a dry reagent compartment 10 and a liquid reservoir 20. In a preferred embodiment, the cartridge 1 is clamshell shaped, consisting of two halves 101, 102 joined by a hinge mechanism 107. The first half 102 contains the dry reagent compartment 10, and the second half 101 contains the liquid reservoir 20. The cartridge case 1 can be composed of a solid material that is relatively impermeable to the liquid contained in the liquid reservoir, such as polymers (e.g. PET, PP, LDPE, HDPE, PVC), ceramics (e.g. glass, alumina), or a composite of materials (e.g. fiberglass). The cartridge 1 can incorporate a diffusion limiting membrane, such as low-density polyethylene, polypropylene, nylon, or others 105.

In an embodiment, the cartridge 1 has a hinge mechanism 107 connecting the liquid reservoir 20 and the dry reagent compartment 10 that enables the two components to be combined into one compartment when they are in contact with each other. The hinge mechanism 107 facilitates the alignment of the two halves 101, 102 after a fluidic barrier 105 is removed, and further helps eliminate air bubbles on the surface of an electrochemical sensor 121 (not shown in FIG. 1) by smoothly moving the liquid reservoir 20 over the surface of the electrochemical sensor 121.

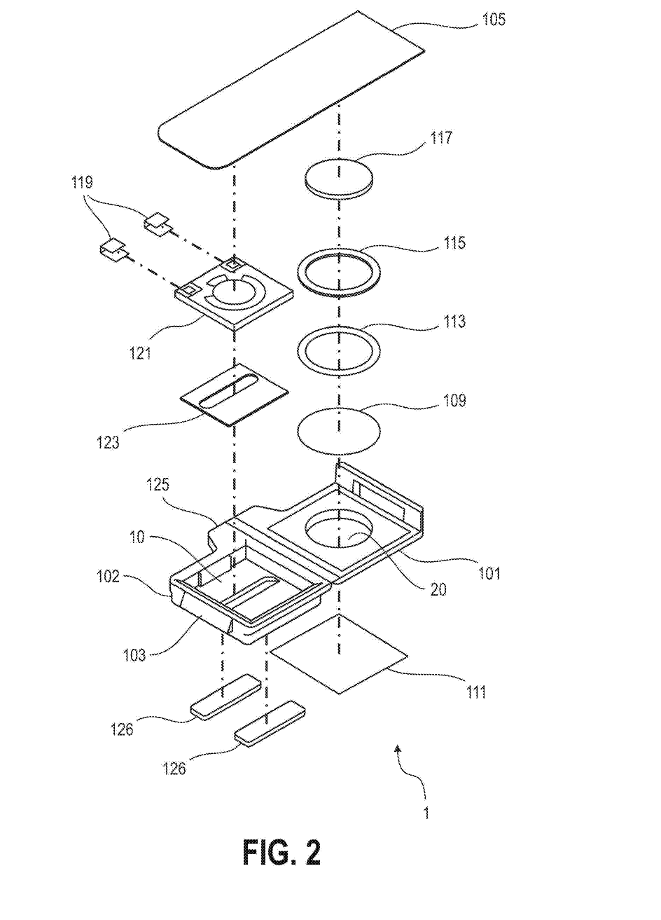

As further shown in the exploded view of FIG. 2, the cartridge 1 includes several components located inside the cartridge case 1. In the first half 102 of the cartridge 1, a pressure-sensitive adhesive (PSA) 123 is placed at the bottom of the dry reagent compartment 10. The electrochemical sensor 121 are placed on the top of the PSA, attached with a pair of electrical connections 119. The dry reagent compartment 10 contains dry reagents such as lyophilized enzymes (not shown) as part of a biosensor used in the detection of a target analyte (not shown) from secretions of the subject using the cartridge 1. Lyophilized enzymes can be alcohol oxidase, glucose oxidase, urate oxidase, or others known to those skilled in the art. The dry reagents can further include lyophilized antibodies, enzymes, organic compounds, or dried inorganic compounds. A pair of magnetic alloys 126 is attached to the bottom of the second half 101 of the cartridge 1. The liquid reservoir 20 of the cartridge 1 is defined by a pressure-sensitive adhesive (PSA) 113, a gasket 115, gels 117, and two biofilms 109, 111 that enclose the liquid reservoir 20 in the second half 101 of the cartridge 1. The liquid (not shown) inside the liquid reservoir 20 may include water, ethanol, solvents, air, hydrogels, ionic liquids, or combinations thereof. The liquid may also contain buffering compounds such as phosphate buffered saline, tris buffer, or other pH-controlled solutions known to those skilled in the art.

The liquid reservoir 20 can include hydrogel such as poly-ethylene-glycol, agarose, or other substances known to those skilled in the art for use in transdermal analyte sensors. In an embodiment, there is an air gap (not shown) inside the liquid reservoir 20 that improves the response time of the electrochemical sensor 121, and reduces pressure fluctuations caused by the user's skin and motion from use of the cartridge 1.

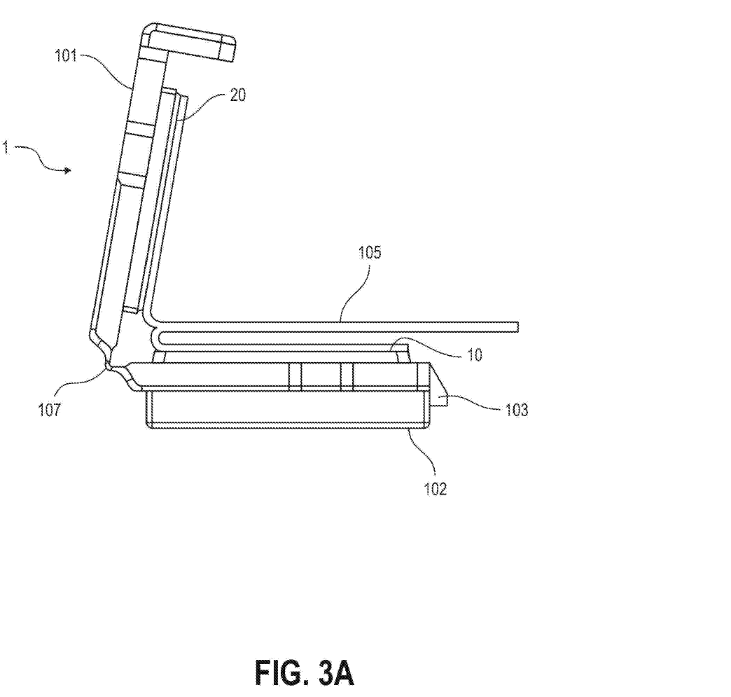

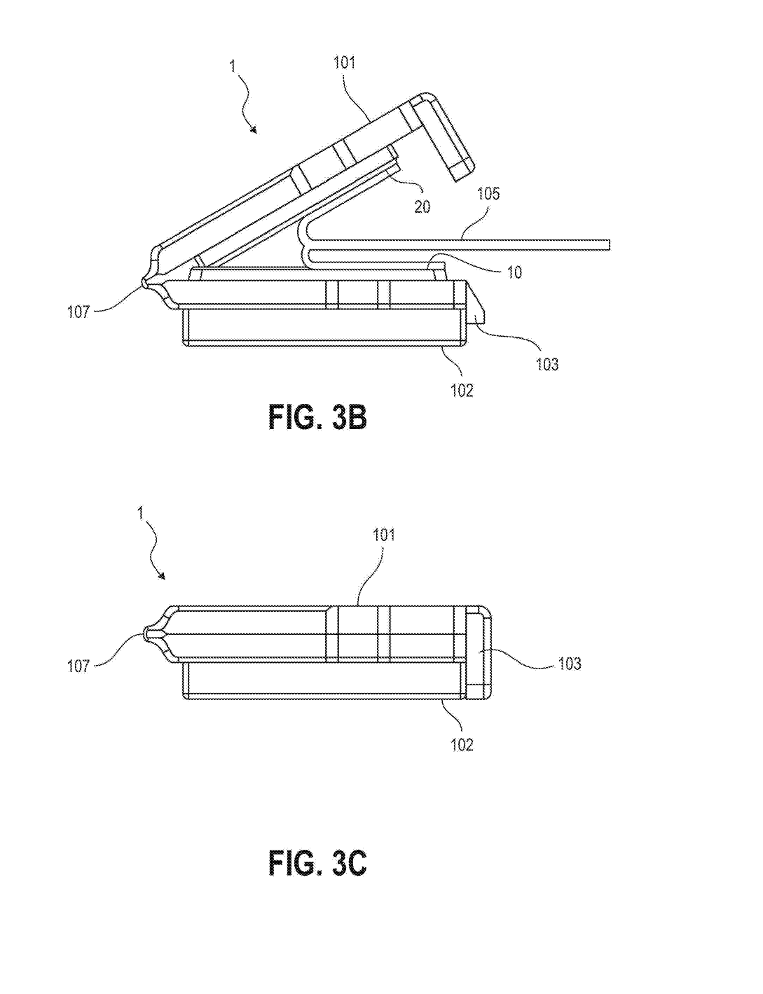

Referring now to FIGS. 3A to 3C, the cartridge 1 according to the present invention has a user-actuated mixing mechanism. The liquid in the liquid reservoir 20 is separated from the dry reagent located inside the dry reagent compartment 10 by a fluidic barrier 105, as shown in FIG. 3A. In an embodiment, the fluidic barrier 105 is a membrane composed of plurality of layers including, but not limited to, low-density polyethylene (LDPE), aluminum foil, wax, and acrylic adhesive, or other polymer or non-polymer materials. The fluidic barrier membrane 105 is attached in a way to enclose the liquid reservoir 20 and the dry reagent compartment 20. Means of attachment of the fluidic barrier membrane 105 include by thermal heat-seal, adhesive method, chemical bonds or methods familiar to those skilled in the art. As shown in FIG. 3A, the fluidic barrier 105 is folded and secured in the cartridge 1 by fastening with the liquid reservoir 20 and the dry reagent compartment 10 in the cartridge 1. When the fluidic barrier 105 is displaced, such as by a user pulling the membrane 105 out of the case as shown in FIG. 3B, the cartridge 1 tends to close the cartridge 1. Upon closure of cartridge 1, the first half 102 of the cartridge 1 is in close proximity with the second half 101 of the cartridge 1. When the fluidic barrier 105 is fully displaced from the cartridge case 101, as shown in FIG. 3C, the liquid reservoir 20 and the dry reagent compartment 10 are connected with each other to form a single mixing compartment. Hence, the removal of the fluidic barrier 105 actuates the mixing mechanism, allowing the liquid to make a direct contact with the dry reagent, and to hydrate the dry reagent in the dry reagent compartment 10. In an embodiment, the mixing mechanism of the dry reagents with the liquid includes passive diffusion, active convection, or other methods known to those skilled in the art.

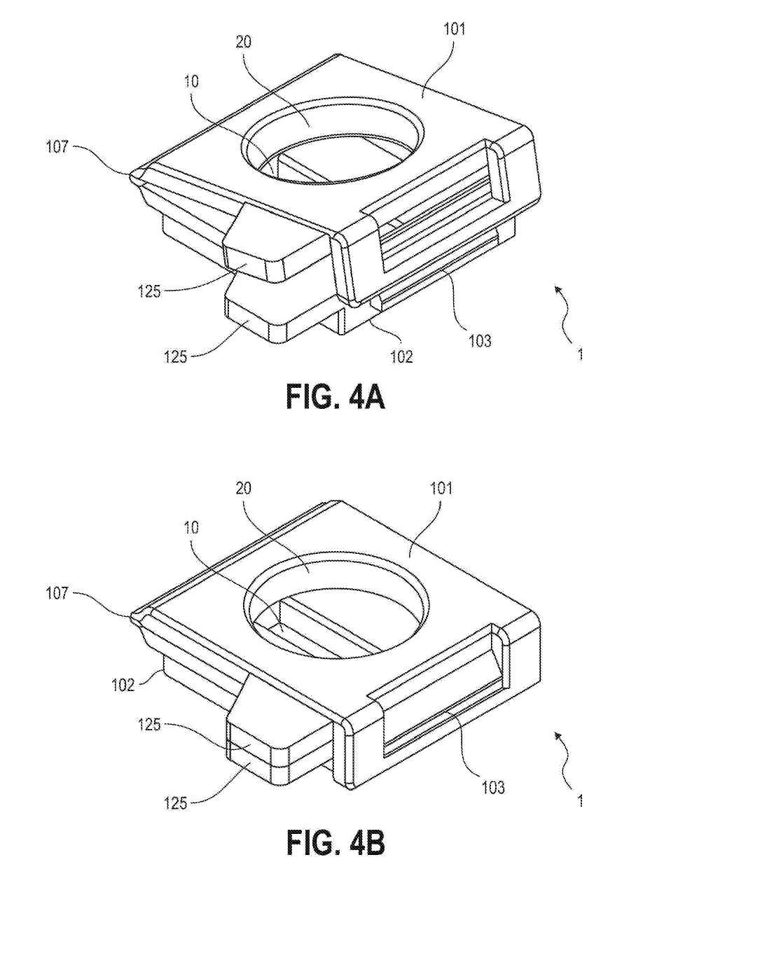

In a preferred embodiment, the cartridge 1 has a snap feature 103 that holds the liquid reservoir 20 onto the dry reagent compartment 10, as shown in FIGS. 4A and 4B. The snap feature 103 keeps the clamshell cartridge case 101 closed and keeps the liquid reservoir 20 aligned with the dry reagent compartment 10, thereby allowing the liquid in contact with the dry reagents once the user-actuated mixing mechanism is activated. In some embodiments, the snap feature 103 is important to the sensing capabilities that utilize electrical conduction through the combined mixing compartment as part of the sensing process. In a preferred embodiment, the snap feature 103 also provides tactile feedback to a user so that the user knows when they have applied sufficient compressive forced to close the cartridge 1.

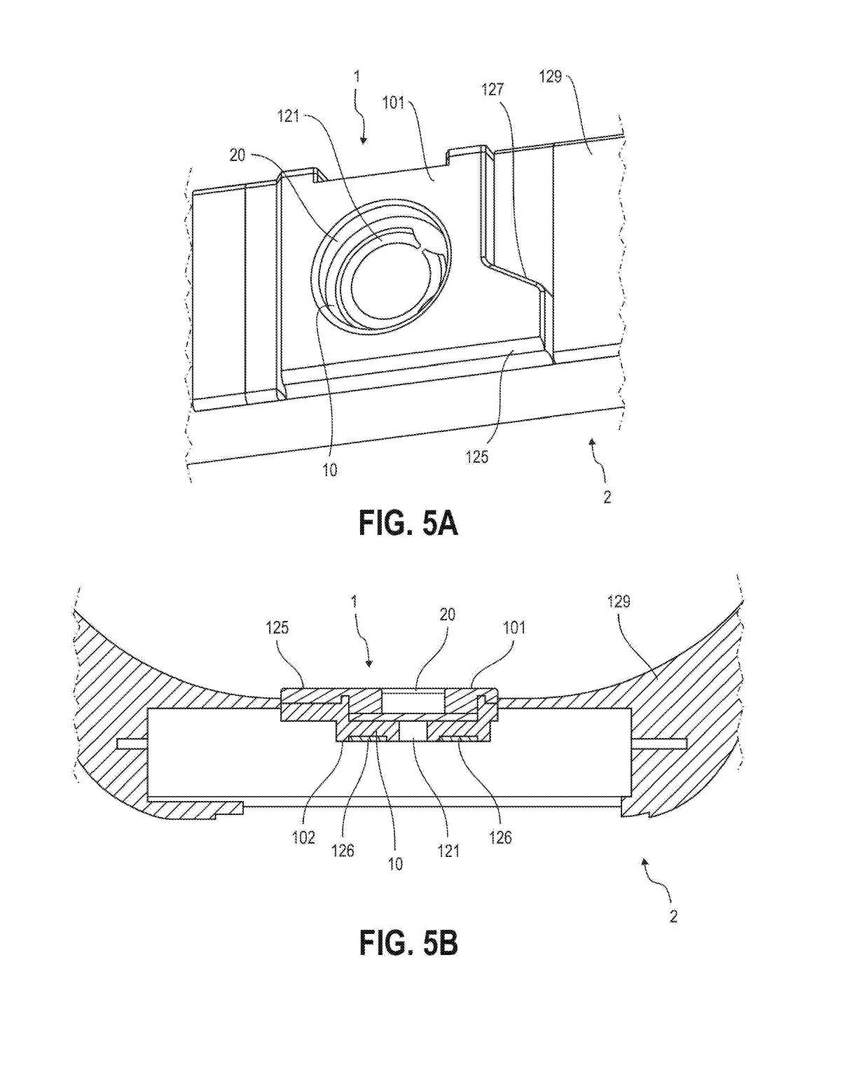

Referring again to FIGS. 4A and 4B, the cartridge 1 has a fin shaped protrusion 125. In a preferred embodiment, the fin shaped protrusion 125 is a protrusion 125 on the cartridge 1 that breaks symmetry of the cartridge 1, allowing the user to orient the cartridge 1. This further allows the user to engage the fin shaped protrusion 125 of the cartridge 1 with a recess 127 on a wristband 129 of the transdermal analyte sensing device 2. The fin shaped protrusion 125 allows the user to easily place the cartridge 1 in a corrected orientation into the enclosure of the wristband 129. As shown in FIG. 5A, the protrusion 125 is engaged with the recess 127 in the enclosure of the wristband 129. When placed on the wristband, the cartridge 1 can remain in direct and unobstructed contact with the test subject while analyzing the target analyte.

As shown in FIG. 5B, the cartridge 1 is placed into the enclosure of the wristband 129. In a preferred embodiment, the cartridge includes a pair of magnetic alloys 126 that allows the cartridge to attach to permanent magnets (not shown) on the wristband 129. The fin shaped protrusion 125 facilitates alignment of the cartridge 1 and the wristband 129.

An example of the use of an embodiment invention is as follows. The dry reagent compartment 10 is enclosed in the transdermal analyte sensing device 2. Once the user removes the fluidic barrier 105, the clamshell-shaped cartridge 1 is closed. The closure of the cartridge 1 causes the first half 102 and the second half 101 of the cartridge 1 to come into contact with each other. In this example, the cartridge 1 is intended for use in transdermal analyte sensing devices 2. To transdermally measure target analyte (i.e. alcohol) of the test subject, the cartridge 1 is engaged with the wristband 129 so that the target analyte can diffuse from secretions of the user, such as sweat or insensible perspiration, into the liquid reservoir 20, and further hydrate and mix with the dry reagent (not shown) inside the cartridge 1.

In a preferred embodiment, the transdermal analyte sensing device 2 includes electrical connections 18 attached to the electrochemical sensor 121 in the dry reagent compartment 10. Hydrated reagents in the cartridge 1 can then be measured by the electrochemical sensor 121 to detect alcohol level of the test subject. For example, hydrated alcohol oxidase can be used for the detection of ethanol diffused from secretions of the test subject in the cartridge 1.

While illustrative embodiments of the invention have been described in detail above, it is to be understood that the appended claims are intended to be construed to include variations of the present invention.

What is claimed is:

1. A transdermal analyte sensing device comprising:
a cartridge, said cartridge having a first and a second compartment, wherein each said compartment stores at least one first component and at least one second component separately;
a user-actuated mixing mechanism, for combining said at least first component and said at least second component.

2. The device of claim 1, wherein said at least one first component further contains a dry reagent.

3. The device of claim 2, wherein said dry reagent is lyophilized enzyme.

4. The device of claim 3, wherein said lyophilized enzyme is alcohol oxidase.

5. The device of claim 1, wherein said at least one second component further comprises a liquid reagent.

6. The device of claim 1, wherein said user-actuated mixing mechanism further comprises at least one fluidic barrier, said at least one fluidic barrier capable of separating said first compartment from said second compartment.

7. The device of claim 1, wherein said cartridge has a snap feature capable of keeping said first compartment in contact with said second compartment.

8. The device of claim 7, wherein said cartridge is clamshell shaped.

9. The device of claim 1, wherein said first compartment further contains an electrochemical sensor.

10. The device of claim 9, wherein said electrochemical sensor is Prussian Blue.

11. A transdermal analyte sensing device, comprising
a cartridge having
a dry reagent compartment,
a liquid reservoir, and
at least one fluidic barrier, said fluidic barrier capable of separating said dry reagent compartment from said liquid reservoir;
said dry reagent compartment has at least one lyophilized enzyme and said liquid reservoir has at least one liquid reagent;
said liquid reservoir capable of receiving a target analyte from secretions of a subject;
wherein when said at least one fluidic barrier is removed from said cartridge, said dry reagent compartment is in contact with said liquid reservoir such that said at least one lyophilized enzyme is capable of reacting with said target analyte.

12. The transdermal analyte sensing device of claim 11, wherein said at least one lyophilized enzyme is alcohol oxidase.

13. The transdermal analyte sensing device of claim 11, wherein said at least one liquid reagent is agarose hydrogel.

14. The transdermal analyte sensing device of claim 11, wherein said target analyte is ethanol.

15. The transdermal analyte sensing device of claim 11 further comprising a snap feature, said snap feature capable of keeping said dry reagent compartment sealed with said liquid reservoir.

16. The transdermal analyte sensing device of claim 11, wherein said dry reagent compartment further comprises an electrochemical sensor, said electrochemical sensor capable of reacting with said lyophilized enzyme and said target analyte.

17. The transdermal analyte sensing device of claim 16, wherein said electrochemical sensor is Prussian Blue.

18. A method for actuating a mixing mechanism, said method comprising:
removing at least one fluidic barrier from a clamshell-shaped cartridge;
connecting a dry reagent compartment with a liquid reservoir when said at least one fluidic barrier is removed;
wherein at least one lyophilized enzyme in said dry reagent compartment is mixed with at least one liquid reagent in said liquid reservoir.

19. The method of claim 18, wherein the mixture of said at least one lyophilized enzyme and said at least one liquid reagent in said cartridge is capable of reacting with a target analyte from secretions of a subject.
